(19) United States
(12) Patent Application Publication
(10) Pub. No.: US 2025/0073159 A1
(43) Pub. Date: Mar. 6, 2025

(54) Composition for a Krill-Powder-Based Skin Care Product

(71) Applicant: Paul Michael David Pierre, Davie, FL (US)

(72) Inventor: Paul Michael David Pierre, Davie, FL (US)

(21) Appl. No.: 18/726,354

(22) PCT Filed: Jan. 3, 2023
(86) PCT No.: PCT/US2023/010046

§ 371 Date: Jul. 2, 2024

Related U.S. Application Data
(60) Provisional application No. 63/296,117, filed on Jan. 3, 2022.

(51) Int. Cl. A61K 8/98; A61K 8/02; A61K 8/19; A61K 8/65; A61K 8/73; A61K 8/92; A61K 8/9794; A61Q 5/00; A61Q 19/00

(52) U.S. Cl. CPC A61K 8/987; A61K 8/022; A61K 8/19; A61K 8/65; A61K 8/735; A61K 8/922; A61K 8/9794; A61Q 5/00; A61Q 19/00

(57) ABSTRACT

A composition for a krill-powder-based skin care product and a method of use thereof rejuvenates the skin and helps maintain a healthy skin and a healthy integumentary system. The composition includes a quantity of krill powder, a quantity of hyaluronic-acid powder, a quantity of calcium-benzoate clay powder, a quantity of aloe-vera powder, a quantity of vitamin-A palmitate powder, and a quantity of colloidal silver. The quantity of krill powder, the quantity of hyaluronic-acid powder, the quantity of calcium-benzoate clay powder, the quantity of aloe-vera powder, the quantity of vitamin-A palmitate powder, and the quantity of colloidal silver are homogeneously mixed into a final powder compound that is non-hydrated. The final powder compound can be rehydrated at the time of application to increase the shelf life of the composition. Further, the composition can be infused together with potential additional ingredients such as retinoid, gelatin, and liquid solvents.

The current application is a Patent Cooperation Treaty (PCT) application and claims a priority to a U.S. provisional application Ser. No. 63/296,117 filed on Jan. 3, 2022.

### FIELD OF THE INVENTION

The present invention relates generally to krill powder formulations and beauty care products. More specifically, the present invention is a non-hydrated krill-powder-based skin care product that can be rehydrated before application.

### BACKGROUND OF THE INVENTION

The cosmetic industry is constantly evolving with new products and combinations of ingredients designed to provide various health benefits to the user's skin. For example, many facemasks and topical creams contain various ingredients designed to hydrate and repair the skin. However, these products overlook the various other potential benefits that can be provided to the skin through a facemask. Further, many facemasks and topical creams are designed, distributed, and sold in a hydrated form. The hydrated form starts a chemical reaction between the various ingredients being used, lowering the beneficial properties of the various ingredients over time, and limiting the amount of time the user must use the product before the product becomes ineffective.

Therefore, an objective of the present invention is to provide a krill-powder-based skin care product to help rejuvenate and maintain healthy skin and hair. The present invention provides the user with a skin care product that can repair and maintain both skin and muscle tissue using bioactive peptides naturally present in krill powder. Another objective of the present invention is to provide a krill-powder-based skin care product that can be rehydrated before application. The present invention can be combined with water or other appropriate fluid base to properly rejuvenate and maintain healthy skin and tissue. By providing a dehydrated product, the present invention also has an increased shelf life. Additional features or benefits of the present invention are further discussed in the sections below.

### SUMMARY OF THE INVENTION

The present invention is a composition for a krill-powder-based skin care product to help with maintaining healthy skin or integumentary system. The present invention seeks to provide users with a skin care product that can hydrate, cleanse, and enrich the skin or integumentary system of the user. To accomplish this, the present invention includes a quantity of krill powder that is rich in naturally occurring, distinct bioactive peptides. Further, calcium-benzoate clay powder is included in the composition to provide a base or foundation for the krill powder, allowing the krill powder to adhere to skin better. Additionally, hyaluronic-acid powder is included in the composition to allow the skin cells to retain moisture better. Thus, a non-hydrated krill powder composition is provided that can be combined with water or other liquid base to properly rejuvenate and maintain healthy skin and tissue. The present invention further comprises additional ingredients such as Vitamin A. In some embodiments, the present invention can also contain a retinoid to provide various anti-aging properties.

### BRIEF DESCRIPTION OF THE DRAWINGS

FIG. 1 is a block diagram of the present invention.

FIG. 2 is a block diagram illustrating the proportions of the present invention.

FIG. 3 is a continuation of FIG. 2.

FIG. 4 is a block diagram of the present invention with additional compositional constituents.

### DETAIL DESCRIPTIONS OF THE INVENTION

All illustrations of the drawings are for the purpose of describing selected versions of the present invention and are not intended to limit the scope of the present invention.

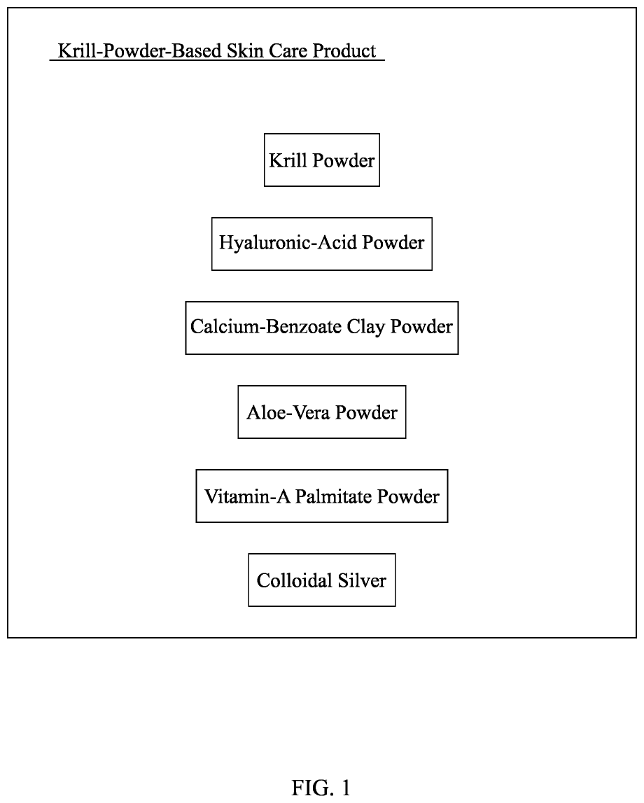

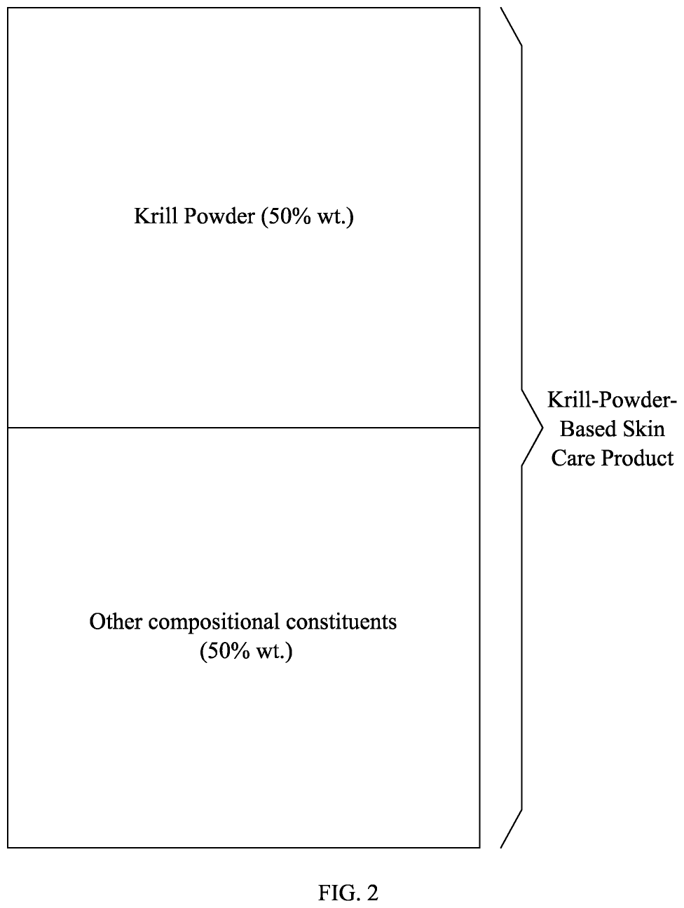

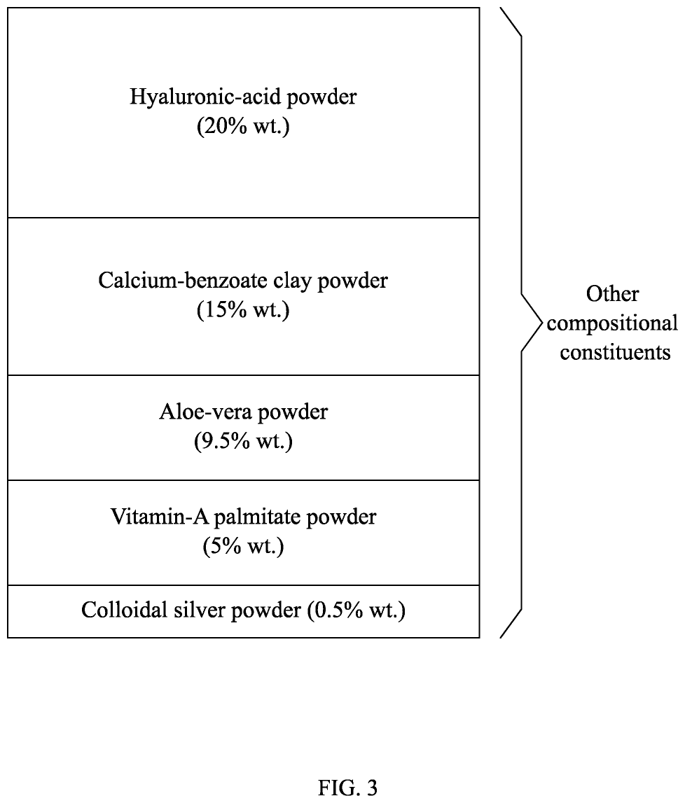

The present invention is a composition for a krill-powder-based skin care product that rejuvenates the skin and helps maintain healthy skin and a healthy integumentary system. The composition of the present invention features bioactive peptides that repair and maintain organ tissue such as skin and hair. As can be seen in FIGS. 1 through 3, the present invention comprises a quantity of krill powder, a quantity of hyaluronic-acid powder, a quantity of calcium-benzoate clay powder, a quantity of aloe-vera powder, a quantity of vitamin-A palmitate powder, and a quantity of colloidal silver. The quantity of krill powder, the quantity of hyaluronic-acid powder, the quantity of calcium-benzoate clay powder, the quantity of aloe-vera powder, the quantity of vitamin-A palmitate powder, and the quantity of colloidal silver are homogeneously mixed into a final powder compound that is non-hydrated. Many of these components allow for the infusion of the various ingredient to produce the final powder compound. The final powder compound can also be rehydrated at the time of application to increase the shelf life of the present invention. Further, the composition of the present invention can be infused with additional ingredients such as retinoid, vitamins, minerals, and a liquid solvent such as water.

In the preferred embodiment, the main ingredient of the composition of the present invention is the quantity of krill powder. The quantity of krill powder contains high levels of distinct, animal-based bioactive peptides. The bioactive peptides are essentially tiny proteins that can easily pass through the skin to repair and maintain skin, organ, and muscle tissue. The krill powder properly rejuvenates and maintains healthy skin and tissue wherever the present invention is applied. These distinct bioactive peptides are similar to collagen peptides commonly found in other cosmetic products, however, the distinct bioactive peptides found in krill powder provide better or more robust benefits to the user unless the user has an allergy to the protein. The highly complex bioactive peptides trigger the healing and development processes of the skin, hair, and surrounding muscles or tissue. Further, the naturally occurring astaxanthin, amino acids, and Omega-3 phospholipids (including docosahexaenoic acid [DHA] and eicosapentaenoic acid [EPA]) in the quantity of krill powder may further assist with various health benefits such as skin, hair, and muscle health to a degree. Furthermore, in some embodiments of the present invention, the quantity of krill powder may be partially or fully hydrolyzed. This means the peptide bonds have been broken down into more easily absorbable amino acids. This lends the benefits of both intact peptide bonds and the amino acids that form the bonds to better heal skin and muscle.

Further, the quantity of calcium-benzoate clay powder is infused with the quantity of krill powder to provide a base or foundation for the krill powder. The quantity of calcium-benzoate clay powder contains various detoxifying properties to ensure the health of the user's skin as well as to facilitate the adhering of the quantity of krill powder to the skin. The quantity of calcium-benzoate clay powder is used to facilitate the adhesion of the quantity of krill powder to the skin surface as well as to facilitate the binding of the various ingredients to homogenize together once mixed with a liquid solvent. The quantity of calcium-benzoate clay powder is commonly used in cosmetics and is a natural and safe binder, but other binder ingredients can be used such as gelatin or gelatin powder. Further, the quantity of hyaluronic-acid powder allows the skin cells to better retain moisture. The quantity of hyaluronic-acid powder allows the bioactive peptides to function and work better to better maintain and hydrate the user's skin. In the dry form, quantity of hyaluronic-acid powder is not chemically active. The quantity of hyaluronic-acid powder needs to be combined with a liquid solvent. Further, the entire composition also needs to be combined with a liquid solvent to be chemically active. Similarly, the quantity of aloe-vera powder provides additional skin-friendly hydrating properties. The quantity of aloe-vera powder also helps relieve symptoms of various skin ailments. Furthermore, the quantity of vitamin-A palmitate powder (a retinoid) and the quantity of colloidal silver both aid in skin care. The quantity of vitamin-A palmitate powder helps increase or stimulate the production of collogen as vitamin-A palmitate is a retinoid which aids in reducing undesirable skin wrinkles. The quantity of colloidal silver aids in skin care by reducing inflammation on the skin. Other retinoids, binding agents, and commonly used ingredients for integumentary health or maintenance may be used in place of the existing ingredients.

In the preferred embodiment, the composition for a krill-powder-based skin care product is properly proportioned to enhance the health benefits of each individual ingredient as well as the individual ingredients aiding the peptides found in krill powder to deliver to the skin and tissue for tissue repairs. As can be seen in FIGS. 1 through 3, the quantity of krill powder is approximately 50 percentage by weight (wt. %) of the final product compound, with an error range of up to ±10 wt. %. The quantity of hyaluronic-acid powder is approximately 20 wt. % of the final product compound, with an error range of up to ±5 wt. %. The quantity of calcium-benzoate clay powder is approximately 15 wt. % of the final product compound, with an error range of up to ±2.5 wt. %. The quantity of aloe-vera powder is approximately 9.5 wt. % of the final product compound, with an error range of up to ±1.25 wt. %. The quantity of vitamin-A palmitate powder is approximately 5 wt. % of the final product compound, with an error range of up to #0.5 wt. %. Further, the quantity of colloidal silver is approximately 0.5 wt. % of the final product compound, with an error range of up to ±0.25 wt. %.

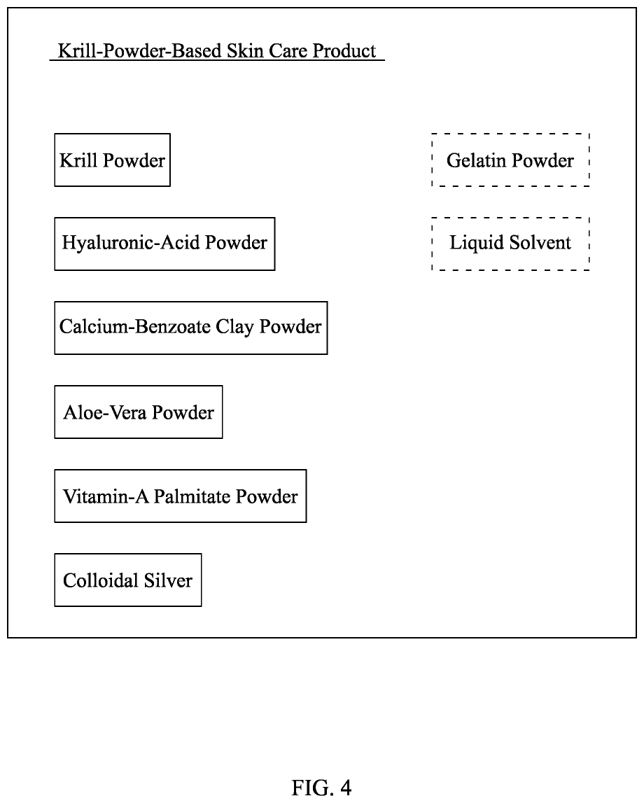

In some embodiments, the composition of the present invention may further comprise a quantity of gelatin powder. As can be seen in FIG. 4, the quantity of gelatin powder can serve as a conditioner and moisturizer to address various skin conditions such as wrinkles and contour deficiencies. Moreover, the quantity of gelatin powder acts as a binder to better adhere ingredients together and with the skin or hair when a liquid solvent is introduced. To do so, the quantity of gelatin powder is homogeneously mixed into the final product compound. Further, the composition of the present invention primarily contains these ingredients infused with each other in a non-hydrated form. The non-hydrated form of the present invention allows for the present invention to maintain a longer shelf life to ensure that the ingredients properly provide the benefits to the user at the time of application in an optimal way.

In another embodiment, the present invention can be packaged in a hydrated form that is ready to use out of the packaging. As can be seen in FIG. 4, the composition of the present invention may further comprise a quantity of liquid solvent that reconstitutes the non-hydrated form of the final product compound. The final product compound is mixed into the quantity of liquid solvent to provide the composition of the present invention in a hydrated form. The quantity of liquid solvent may be different appropriate liquids including, but not limited to, a quantity of mineral water, a quantity of distilled water, a quantity of liquid fat, or a quantity of liquid oil, or a combination thereof. The quantity of liquid oil may include, but is not limited to, a quantity of olive oil, a quantity of coconut oil, or a combination thereof. The hydrated form of the present invention can be used as a hydrated facemask that can be readily applied. Further, in another embodiment, the present invention may further include one or more retinoid ingredients. The one or more retinoid ingredients provide the composition of the present invention with more anti-aging properties that benefit the user's skin.

Although the invention has been explained in relation to its preferred embodiment, it is to be understood that many other possible modifications and variations can be made without departing from the spirit and scope of the invention.

1. A composition for a krill-powder-based skin care product comprising:
a quantity of krill powder;
a quantity of hyaluronic-acid powder;
a quantity of calcium-benzoate clay powder;
a quantity of aloe-vera powder;
a quantity of vitamin-A palmitate powder;
a quantity of colloidal silver; and
the quantity of krill powder, the quantity of hyaluronic-acid powder, the quantity of calcium-benzoate clay powder, the quantity of aloe-vera powder, the quantity of vitamin-A palmitate powder, and the quantity of colloidal silver being homogeneously mixed into a final powder compound.

2. The composition for a krill-powder-based skin care product claimed in claim 1 comprising:
the quantity of krill powder being approximately 50 percentage by weight (wt. %) of the final product compound.

3. The composition for a krill-powder-based skin care product claimed in claim 1 comprising:
the quantity of hyaluronic-acid powder being approximately 20 wt. % of the final product compound.

4. The composition for a krill-powder-based skin care product claimed in claim 1 comprising:
the quantity of calcium-benzoate clay powder being approximately 15 wt. % of the final product compound.

5. The composition for a krill-powder-based skin care product claimed in claim 1 comprising:
the quantity of aloe-vera powder being approximately 9.5 wt. % of the final product compound.

6. The composition for a krill-powder-based skin care product claimed in claim 1 comprising:
the quantity of vitamin-A palmitate powder being approximately 5 wt. % of the final product compound.

7. The composition for a krill-powder-based skin care product claimed in claim 1 comprising:
the quantity of colloidal silver being approximately 0.5 wt. % of the final product compound.

8. The composition for a krill-powder-based skin care product claimed in claim 1 comprising:
a quantity of gelatin powder; and
the quantity of gelatin powder being homogeneously mixed into the final product compound.

9. The composition for a krill-powder-based skin care product claimed in claim 1 comprising:
a quantity of liquid solvent; and
the final product compound being mixed into the quantity of liquid solvent.

10. The composition for a krill-powder-based skin care product claimed in claim 9, wherein the quantity of liquid solvent is a quantity of mineral water.

11. The composition for a krill-powder-based skin care product claimed in claim 9, wherein the quantity of liquid solvent is a quantity of distilled water.

12. The composition for a krill-powder-based skin care product claimed in claim 9, wherein the quantity of liquid solvent is a quantity of liquid fat.

13. The composition for a krill-powder-based skin care product claimed in claim 9, wherein the quantity of liquid solvent is a quantity of liquid oil.

14. The composition for a krill-powder-based skin care product claimed in claim 13, wherein the quantity of liquid oil is a quantity of olive oil.

15. The composition for a krill-powder-based skin care product claimed in claim 13, wherein the quantity of liquid oil is a quantity of coconut oil.

16. The composition for a krill-powder-based skin care product claimed in claim 13, wherein the quantity of krill powder is configured to maintain and rejuvenate human hair.

17. The composition for a krill-powder-based skin care product claimed in claim 13, wherein the quantity of krill powder is configured to maintain and rejuvenate human skin.
